(19) United States
(12) Patent Application Publication
(10) Pub. No.: US 2010/0087006 A1
(43) Pub. Date: Apr. 8, 2010

(54) Use of fluorescent protein in cyanobacteria and algae for improving photosynthesis and preventing cell damage

(75) Inventors: Jonathan Gressel, Rehovot (IL); Doron Eisenstadt, Haifa (IL); Daniella Schatz, Givataim (IL); Shai Einbinder, Hofit (IL); Shai Ufaz, Givat Ada (IL)

Correspondence Address: John Dodds, 1707 N St. NW, Washington, DC, 20036, US

(73) Assignee: TransAlgae Ltd, Rehovot (IL)

(21) Appl. No.: 12/586,185

(22) Filed: Sep. 18, 2009

Related U.S. Application Data
(60) Provisional application No. 61/192,447, filed on Sep. 18, 2008.

(51) Int. Cl. C12N 15/74; C12N 1/21; C12N 1/13

(52) U.S. Cl. 435/471; 435/252.3; 435/257.2

(57) ABSTRACT

This disclosure provides a method to reduce cell damage caused by near UV light absorption of algal or cyanobacterial cultures. The algal or cyanobacterial cells are transformed to express one or more fluorescent proteins, that absorb the harmful UV or near UV wavelengths and emits wavelengths that are photosynthetically more active. The photosynthetic pigments of the transgenic algal or cyanobacterial cell culture will then absorb the photosynthetically active light emitted by the fluorescent proteins. Accordingly the harmful effects of the UV and near UV radiation are reduced and the photosynthetic activity of the algal or cyanobacterial cells is improved.

### PRIORITY

This application claims priority of U.S. provisional application No. 61/192,447 filed on Sep. 19, 2008.

### SEQUENCE LISTING

This application contains sequence data provided on a computer readable diskette and as a paper version. The paper version of the sequence data is identical to the data provided on the diskette.

### FIELD OF THE INVENTION

This invention is related to the field of plant molecular biology. More specifically the invention is related to the field of improving photosynthetic efficiency and reducing cell-damage caused by near ultraviolet light by transgenically integrating fluorescent protein encoding genes into algae and cyanobacteria.

### BACKGROUND OF THE INVENTION

Bioreactors for photosynthetic organisms have been proposed for the production of pharmaceuticals, natural pigments, single cell proteins, secondary metabolites and more recently for mass culture of microalgae and cyanobacteria that contain high oil concentrations for producing biodiesel and for other uses, as well as other co-products. Many problems are to be overcome before bioreactors can be efficiently used for biodiesel production (Chisti 2007). Sunlight contains near-UV wavelengths that cause cell damage and can reduce biomass yield, as well as raise the temperature of the culture medium to above optimum temperature. Many cyanobacteria naturally synthesize compounds that can act as UV blockers (Sinha and Hader 2007), but these compounds dissipate the absorbed energy as heat, and thus do not enhance photosynthesis. Dyes absorbing light in the near UV wavelength region have been thought to be effective in enhancement of algal growth, but the dyes proved toxic to the algae. Despite these problems, Prokop et al. (1984) stated that incorporation of dyes into the media of algae suspensions does in fact provide additional light source and enhance growth.

### SUMMARY OF THE INVENTION

In this disclosure we solve the problem of near-UV light causing cell damage and reducing biomass with a novel approach. Namely, our approach is to use proteinaceous fluorescent pigments that absorb light at wavelengths not used efficiently by the plants and emit light at favorable wavelengths for algal growth and photosynthetic yields. Endogenously including natural, biological pigments into a photosynthetic organism where they would be much more efficient has never been envisaged before.

Some organisms possess a great variety of compounds that absorb light of many colors and fluoresce the light at longer wavelengths. Their visual effects are either due to the intricate ultrafine physical organization of tissues that results in differential scattering of the incoming light, or to the display of specific colored molecules (pigments), or to the combination of both. The pigments are usually small molecules featuring extended conjugated pi-systems in their chemical structure, which endow them with chemical resonance of frequencies residing within the wavelength span of the visible spectrum (400 to 750 nanometers). The green fluorescent protein-like (GFP-like) family are the only known pigments that are essentially encoded by a single gene, since both the substrate for pigment biosynthesis and the necessary catalytic moieties are contained within a single polypeptide chain thus serving both as a substrate and an enzyme. The only external agent required to complete the pigment biosynthesis is molecular oxygen (Heim et al., 1994).

The prototypical GFP from the bioluminescent jellyfish Aequorea victoria and its derivatives and analogs have become important imaging tools in molecular and biological sciences where they are used as cell and protein labels, visible markers of gene expression both by themselves and as fusion proteins for use in cellular physiological studies. Recently, it was discovered that the majority of the bright colors of Anthozoa (i.e. reef corals, anemones and other related organisms) are determined by proteins homologous to GFP. These include fluorescent blue, green, yellow and red proteins and the lower wavelength—fluorescent, purple-blue hues. The discovery of GFP-like proteins in non bioluminescent organisms has greatly expanded multi-color labeling as well as other applications. A variety of fluorescent proteins ranging from cyan to red colors isolated from reef corals are now commercially available and novel varieties are being constantly discovered.

Corals have a symbiotic relationship with dinoflagellate microalgae (zooxanthellae) that live within their endodermal cells. Consequently, corals are highly dependent on sunlight for the photosynthesis of the zooxanthellae from which they derive much of their own energy requirements. By focusing on spectral, microstructural and eco-physiological studies of coral fluorescent proteins in vivo, Salih et al. (2000) proposed that they function in light optimization of coral tissues for photosynthetic requirements of their intracellular microalgal symbionts.

To improve the currently available systems, we use genes encoding native fluorescent proteins or genes encoding, native proteins that have been artificially modified to increase their stability, after they have been adapted to the codon usage of the algae/cyanobacteria used. They are overexpressed in each cell to create a unique and better light regime in the bioreactor. This is achieved by using a fluorescent protein that absorbs light in the near-UV region and emits light in the photosynthetic range of the recipient organism thus enhancing photosynthesis and preventing cell damage caused by short wavelength light. In addition, we also use other native or synthetic genes encoding other fluorescent proteins that absorb light in photosynthetically underutilized wavebands (such as the green wavelengths) and emit light in the photosynthetic range of the recipient organism. These genes are adapted to the codon usage of the algae/cyanobacteria used and overexpressed in each cell. These genes can be expressed in tandem with other genes or used in co-transformations and thereby also be used as selectable markers. Additionally, two or more fluorescent proteins can be introduced into the cells in order to reach optimal photosynthetic efficiency.

Accordingly, this invention provides a method to enhance algal and cyanobacterial photosynthesis and/or prevent cell damage caused by short wavelengths, by the over expression of naturally occurring or synthetic genes encoding fluorescent proteins within the cells. These genes are configured to match the preferred codon usage of the target organism used. The genes can be expressed alone or fused to a specific transit peptide or targeting protein that will lead them to specific locations within the cells. These transgenic algae/cyanobacteria can serve as a platform for further engineering of desired traits when also used as selectable markers.

The method according to this invention can be used for both freshwater and marine photosynthetic organisms.

### A SHORT DESCRIPTION OF THE DRAWINGS

FIG. 1. Action spectra of photosynthetic O2 evolution in Cryptophyta and Chlorophyta (thin black). Excitation spectra of fluorescent protein (thick grey). Emission spectra of fluorescent protein (thick black).

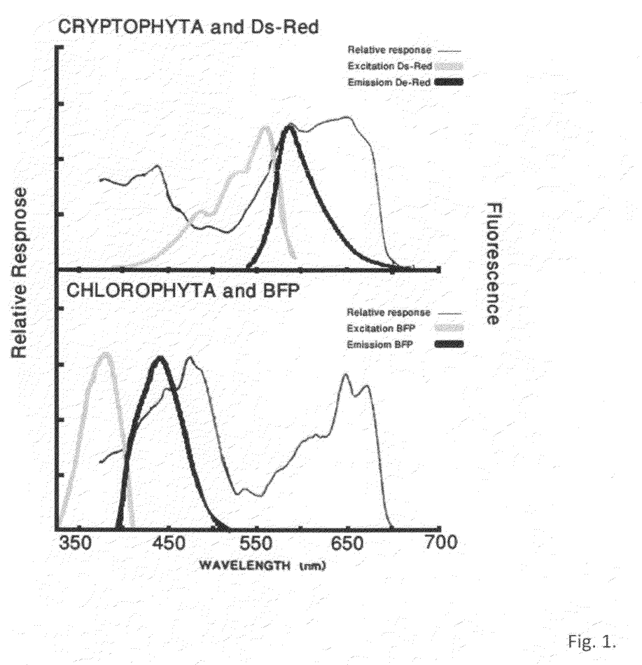

FIG. 2. Plasmid map containing the DNA cassette used to transform the green algae C. reinhardtii, the eustigmatophyte Nannochloropsis oculata and the haptophyte Isochrysis sp. with the blue fluorescent protein (BFP)-azurite gene. The modified coding sequence of BFP-azurite gene was cloned into the BstBI/BamHI sites downstream to the Hsp70A/RbcS2 promoter and RbcS2 first intron and upstream to the 3′ RbcS2 terminator.

FIG. 3. Schematic diagram of the DNA fragment used to transform the cyanobacterium Synechococcus PCC7002 with the blue fluorescent protein (BFP)-azurite gene. The modified coding sequence of BFP-azurite gene according to the Synechococcus PCC7002 codon usage was cloned into the BamHI site of pCB4 downstream to the RbcLS promoter.

FIG. 4. UV LED (light emitting diode) spectrum used for excitation of fluorescent proteins (as specified by supplier, Nichia, Tokyo, Japan)

FIG. 5. PCR screen for BFP containing Chlamydomonas reinhardtii transformants. PCR with BFP specific primers was performed on DNA extracted from 22 colonies grown on selectable medium. The specific primers were designed to amplify a 511 by product. M—marker; 1 to 22—transformants.

FIG. 6. mRNA expression of BFP in Chlamydomonas reinhardtii transformants containing pSI-BFP-Pt. PCR was performed on cDNA synthesized from RNA extracted from 10 selected transgenic colonies. M—marker; -rt—control for DNA contamination; NTC—no template control. The specific primers were designed to amplify a 511 bp product.

### DETAILED DESCRIPTION OF THE INVENTION

Algae and cyanobacteria with biotechnological utility are chosen from among the following, non-exclusive list of organisms

List of Species:

Chlamydomonas reinhardtii, Pavlova lutheri, Isochrysis sp. CS-177, Nannochloropsis oculata CS-179, Nannochloropsis like CS-246, Nannochloropsis salina CS-190, Tetraselmis suecica, Tetraselmis chuii and Nannochloris sp. as representatives of all algae species. Synechococcus PCC7002, Synechococcus WH-7803, Thermosynechococcus elongatus BP-1 as representatives of all cyanobacterial species. The algae come from a large taxonomical cross section of species (Table 1)

TABLE 1
Phylogeny of some of eukaryotic algae used
Phylogeny of eukaryotic algae used

| Genus | Family | Order | Phylum | Sub-Kingdom |
|---|---|---|---|---|
| Chlamydomonas | Chlamydomonadaceae | Volvocales | Chlorophyta | Viridaeplantae |
| Nannochloris | Coccomyxaceae | Chlorococcales | Chlorophyta | Viridaeplantae |
| Tetraselmis | Chlorodendraceae | Chlorodendrales | Chlorophyta | Viridaeplantae |
| Phaeodactylum | Phaeodactylaceae | Naviculales | Bacillariophyta | Chromobiota |
| Nannochloropsis | Monodopsidaceae | Eustigmatales | Heterokontophyta | Chromobiota |
| Pavlova | Pavlovaceae | Pavlovales | Haptophyta | Chromobiota |
| Isochrysis | Isochrysidaceae | Isochrysidales | Haptophyta | Chromobiota |

Phylogeny according to: http://www.algaebase.org/browse/taxonomy/
Note:
Many genes that in higher plants and Chlorophyta are encoded in the nucleus are encoded on the chloroplast genome (plastome) in the Chromobiota red lineage algae (Grzebyk, et al., 2003)

The General Approach for Algae and Cyanobacteria is as Follows:

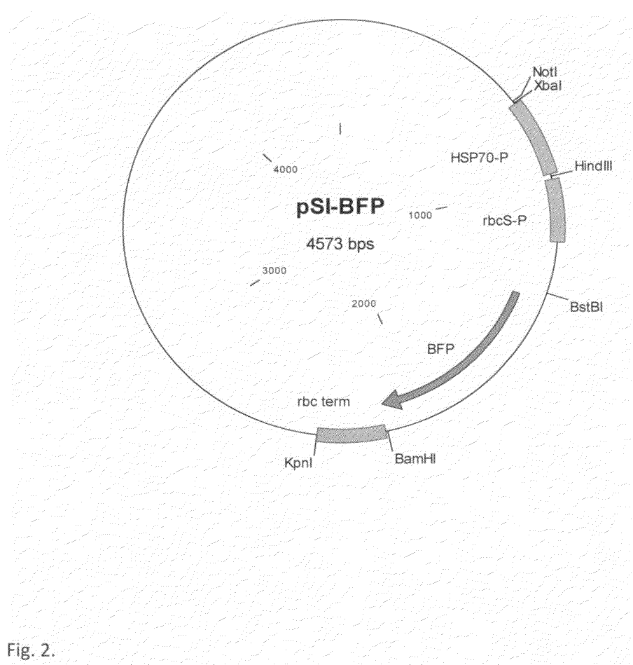

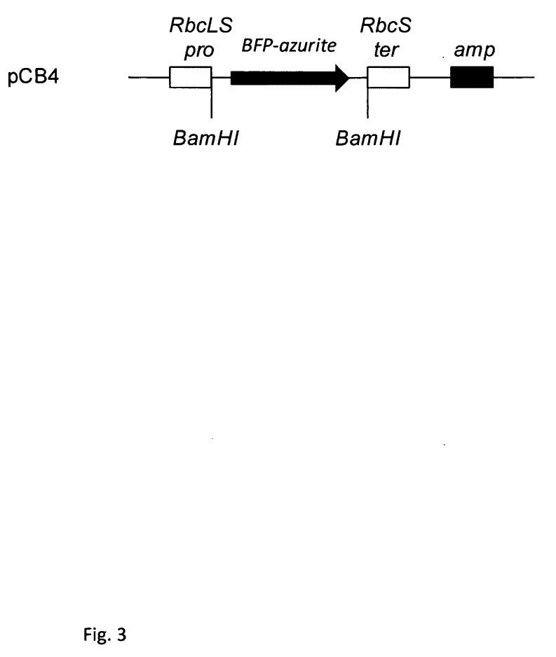

De novo synthesized blue fluorescent protein (BFP)-azurite, A5cDNA (Mena et al., 2006) or other fluorescence proteins such as DsRed, for enhancing algal and cyanobacterial photosynthesis and/or preventing cell damage caused by short wavelengths were cloned under the control of the Hsp70-rbcS2 promoter or other constitutive promoters and 3′rbcS2 terminator for algae (FIGS. 2 and 3). More than one fluorescent protein can be cloned in tandem to achieve stacking, leading to optimal utilization of the total light spectrum reaching the culture. Genes encoding more than one fluorescent protein can be functionally stacked in a sequential manner, or by co-transformation.

The methodologies used in the various steps of enabling the invention are described below:

Transformation of Chlamydomonas

Algae cells in 0.4 ml of growth medium containing 5% PEG MW6000 were transformed with, for example, 1 to 5 μg of the plasmid described in example 1, by the glass bead vortex method (Kindle, 1990). The transformation mixture was then transferred to 10 ml of non-selective growth medium for recovery and incubated for at least 18 h at 25° C. in the light. Cells were collected by centrifugation and plated at a density of 10⁸ cells per 80 mm Petri dish. Transformants were grown on fresh TAP or SGII agar plates containing a selective agent for 7-10 days at 25° C.

Transformation of Marine Algae

I. Electroporation
Fresh algal cultures are grown to mid exponential phase in artificial seawater (ASW)+f/2 media. Cells are then harvested and washed twice with fresh media. After resuspending the cells in 1/50 of the original volume, protoplasts are prepared by adding an equal volume of 4% hemicellulase (Sigma) and 2% Driselase (Sigma) in ASW and are incubated at 37° C. for 4 hours. Protoplast formation is tested by Calcofluor white non-staining. Protoplasts are washed twice with ASW containing 0.6M D-mannitol (Sigma) and 0.6M D-sorbitol (Sigma) and resuspended in the same media, after which DNA is added (10 μg linear DNA for each 100 μl protoplasts). Protoplasts are transferred to cold electroporation cuvettes and incubated on ice for 7 minutes, then pulsed in an ECM830 electroporation apparatus (BTX, Harvard Apparatus, Holliston, Mass., USA). A variety of pulses is usually applied, ranging from 1000 to 1500 volts, 10-20 msec per pulse. Each cuvette is pulsed 5-10 times. Immediately after pulsing the cuvettes are placed on ice for 5 minutes and then the protoplasts are added to 250 μl of fresh growth media (without selector). After incubating the protoplasts for 24 hours in low light at 25° C. the cells are plated onto selective solid media and incubated under normal growth conditions until single colonies appear.

II. Microporation
A fresh algal culture is grown to mid exponential phase in ASW+f/2 media. A 10 ml sample of the culture is harvested, washed twice with Dulbecco's phosphate buffered saline (DPBS, Gibco, Invitrogen, Carslbad, Calif., USA) and resuspended in 250 μl of buffer R (supplied by Digital Bio, NanoEnTek Inc., Seoul, Korea, the producer of the microporation apparatus and kit). After adding 8 μg linear DNA to every 100 μl cells, the cells are pulsed. A variety of pulses is usually needed, depending on the type of cells, ranging from 700 to 1700 volts, 10-40 msec pulse length; each sample is pulsed 1-5 times. Immediately after pulsing, the cells are transferred to 200 μl fresh culture media (without selector). After incubating for 24 hours in low light at 25° C., the cells are plated onto selective solid media and incubated under normal culture conditions until single colonies appear.

III. Particle Bombardment
A fresh algal culture is grown to mid exponential phase in ASW+f/2 media. 24 hours prior to bombardment cells are harvested, washed twice with fresh ASW+f/2 and resuspended in 1/10 of the original cell volume in ASW+f/2. 0.5 ml of each cell suspension is spotted onto the center of a 55 mm Petri dish containing 1.5% agar solidified ASW+f/2 media. Plates are left to dry under normal growth conditions. Bombardment is carried out using a PDS 1000/He biolistic transformation system according to the manufacturer's (BioRad Laboratories Inc., Hercules, Calif., USA) instructions using M10 tungsten powder (BioRadLaboratories Inc.) for cells larger than 2 microns in diameter, and tungsten powder comprised of particles smaller than 0.6 microns (FW06, Canada Fujian Jinxin Powder Metallurgy Co., Markham, ON, Canada) for smaller cells. The tungsten is coated with linear DNA. 1100 or 1350 psi rupture discs are used. All disposables (unless otherwise noted) are supplied by BioRad Laboratories Inc. After transformation the cells are incubated under standard culture conditions for 24 hours, followed by transferring the cells onto selective solid media at a density of 10⁴ cells per 90 mm diameter plates, and incubated under normal growth culture until single colonies appear.

Transformation of Cyanobacteria

For transformation to Synechococcus PCC7002, cells are cultured in 100 ml of BG-1130 Turks Island Salts liquid medium (http://www.crbip.pasteur.fr/fiches/fichemedium.jsp?id=548) at 28° C. under white fluorescent light and subcultured at mid exponential growth. To 1.0 ml of cell suspension containing 2×10⁸ cells, 0.5-1.0 μg of donor DNA (in 10 mM Tris/1 mM EDTA, pH8.0) is added, and the mixture is incubated in the dark at 26° C. overnight. After incubation for a further 6 h in the light, the transformants are selected on BG-11+Turks Island Salts agar plates containing a selection agent until single colonies appear.

Quantification of Transgenic Protein

For quantification of the transgene expression products, proteins are isolated from the algal cells utilizing a buffer containing 750 mM Tris pH 8.0, 15% sucrose (wt/vol), 100 μM β-mercaptoethanol and 1 mM phenylmethylsulfonylfluoride (PMSF). Samples are then centrifuged for 20 min at 13,000×g at 4° C., with the resulting supernatant used in western immunoblotting. Western immunoblotting is carried out as described by Cohen et al. (1998) using a rabbit anti-RCFP polyclonal Pan antibody that detects any of the entire panel of GFP-like reef coral fluorescent proteins (Clontech, Palo Alto, Calif., USA) and an alkaline phosphatase-labeled goat anti-rabbit secondary antibody (Sigma).

Proteins for in vitro BFP assays are prepared in the same fashion except that the crude lysate is centrifuged for 30 min at 40,000×g at 4° C. to remove contaminating thylakoids. Microtiter assays are carried out on volumes of 100 μl with samples diluted in protein extraction buffer. Protein concentrations are determined using Bio-Rad Protein assay reagent (Bio-Rad Laboratories Inc).

RNA Extraction, cDNA Synthesis and Quantitative RT-PCR Analysis

For screening for transgenes expressing high levels of BFP mRNA, total RNA is isolated using either QIAGENS's plant RNeasy Kit (QIAGEN, Hilden, Germany) or the Trizol reagent (Invitrogen, Carlsbad, Calif., USA). cDNA is synthesized using 3 μg total RNA as a template with an oligo-dT primer for algae and a specific 3′primer for cyanobacteria, and SuperScript™ II reverse transcriptase (Invitrogen, Carlsbad, Calif., USA) according to the manufacturer's instructions. Presence of BFP-azurite DNA was tested by PCR using BFP-azurite specific primers (Sequence in example 1). REDTaq DNA polymerase (Sigma) was used for the PCR amplification. A 1 kb DNA ladder was used as DNA size marker (Fermentas, Md., USA).

Photosynthetic Efficiency and Culture Growth

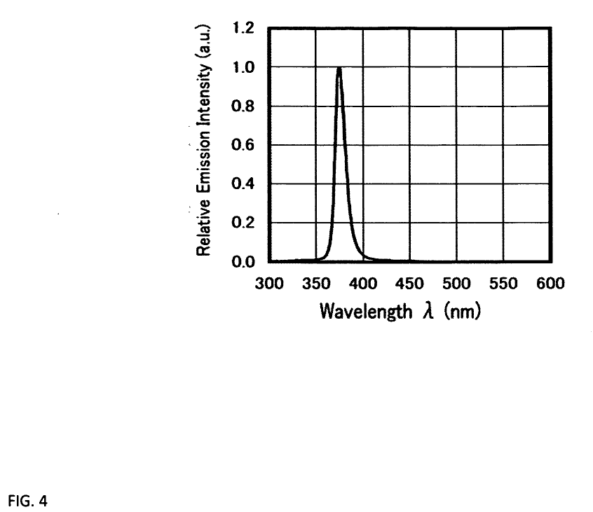

Fluorescent proteins transform high energy, damaging (near-UV) wavelengths into lower energy, longer, less damaging (blue to red) wavelengths. Fluorescent proteins with overlapping excitation and emission spectra, can convert light from any wavelengths (near-UV, green) poorly used by photosynthetic pigments into photosynthetically more active wavelengths. In order to test the hypothesis that cells expressing synthetic genes encoding fluorescent proteins will be more efficient using whole light spectra reaching the culture, cells expressing the BFP-azurite or any other type of fluorescent protein are compared to wild type cells. To assess the contribution of fluorescent proteins to cell photosynthetic efficiency, cells are illuminated with narrow band light with a peak at excitation wavelength of the fluorescent proteins. (e.g. a near-UV LED—light emitting diode) emitting at 375±5 nm (FIG. 4). Photosynthetic activities of the transgenic algae are examined and compared to those of the wild types by measuring oxygen evolution in the light and oxygen consumption in the dark, using Clark type electrodes (Pasco Scientific, Roseville, Calif., USA).

A setup for comparative evaluation of oxygen evolution was built, allowing simultaneous measurements of 8 algal samples illuminated at different intensities and wavelengths. Temperature is maintained using a water-bath with circulator (Model CB 8-30e, Heto Lab Instruments).

Culture Conditions

Cells of eukaryotic marine cultures (e.g. Isochrysis galbana, Phaeodactylum tricornutum and Nannochloropsis sp.) and transformants thereof are cultured on artificial seawater (ASW) medium (Wyman et al., 1985) supplemented with f/2 (Guillard and Ryther, 1962). Marine cultures are grown at 22-25° C. with a 16/8 h light/dark period. Fresh water cultures (e.g. Chlamydomonas reinhardtii) and transformants thereof are cultured photoautotrophically on in liquid medium, using mineral medium as previously described (Harris, 1989), supplemented with 5 mM NaHCO₃, with continuous shaking and illumination at 22° C. Cells of marine cyanobacteria (e.g. Synechococcus PCC 7002) and transformants thereof are cultured in medium BG-11+Turks Island salts liquid medium (http://www.crbip.pasteur.fr/fiches/fichemedium.jsp?id=548). Cyanobacteria are cultured at 25° C. under continuous white light, with constant CO₂-air bubbling.

In order to test the hypothesis that cells expressing synthetic genes encoding fluorescent proteins are more efficient than the wild type capable of using sunlight, we compare algae expressing fluorescent proteins to wild type cells in ambient sunlight.

For example, the growth rate of wild type and BFP-azurite transformants cultured in PAR (photosynthetically active radiation—i.e. 450-750 nm light) and PAR+near-UV is measured using direct cell counts. Culture density is measured daily for a period of ten days. The growth rate of wild type and DsRed transformants cultured in sunlight is measured using direct cell counts. Culture density is measured daily for a period of ten days.

Algae and cyanobacteria expressing fluorescent proteins have increased photosynthetic activity and growth rate compared to the wild type at the tested wider light spectrum containing near-UV.

The invention is now described by means of various non-limiting examples:

### Example 1

### Generation of Eukaryotic Algae Cells Expressing BFP-Azurite

The BFP-azurite sequence (Mena et al., 2006) was artificially synthesized using the published sequence (SEQ ID NO: 1) with modifications according to the codon usage of P. tricornutum (BFP-Pt) (SEQ ID NO: 2) and the green algae C. reinhardtii (BFP-Cr) (SEQ ID NO: 3) and with the addition of BstBI and BamHI restriction sites at its ends. The gene was cloned into pGEM-T vector (Promega, Madison, USA) and then ligated into the BstBI/BamHI restriction sites of pSI103 (Sizova et al., 2001) replacing the aphVIII selectable marker gene, generating the plasmid pSI-BFP. In this plasmid the BFP-azurite gene is under the control of the Hsp70A/RbcS2 promoter and 3′ RbcS2 terminator.

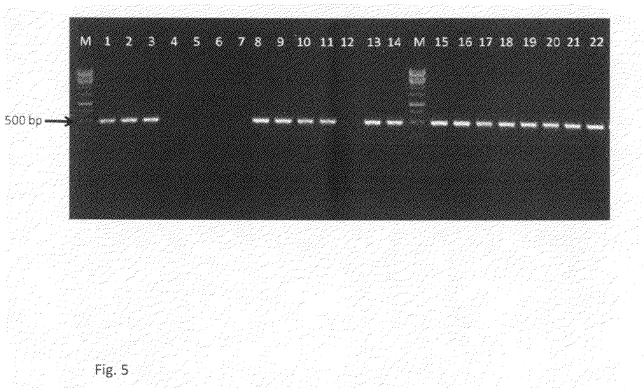

Parental strain C. reinhardtii CC-425 was co-transformed with the pSI-BFP-Pt plasmid and linearized plasmid pJD67, containing the structural gene (ARG7) of the argininosuccinate lyase to complement the arg2 locus (Davies et al. 1994, 1996). C. reinhardtii colonies were selected on TAP medium without arginine. Approximately 35 colonies that grew without arginine were transferred to liquid TAP medium and screened for pSI-BFP construct using PCR with primers (FIG. 5):

BFP-forward primer (SEQ ID NO: 4):
CTGGACGGAGATGTTAATGG
and
BFP-reverse primer (SEQ ID NO: 5):
TCGGAGTGTTCTGCTGATAG.

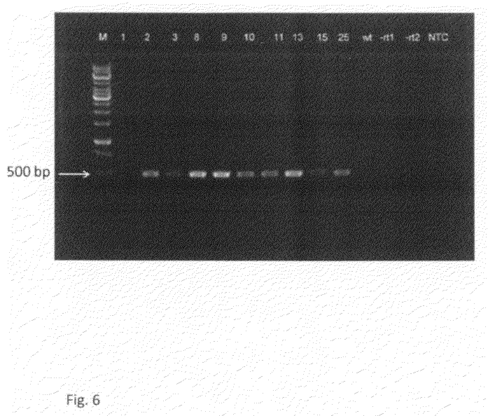

RNA was extracted from positive colonies containing the pSI-BFP construct for BFP expression monitoring by RT-PCR on cDNA using the primers BFP-forward and BFP-reverse (FIG. 6). Colonies expressing the BFP transcript are then screened for BFP expression as described in example 5.

In addition, the pSI-BFP-Pt/Cr plasmid together with pSI-PDS plasmid containing the pds gene (conferring resistance to the phytoene desaturase-inhibiting herbicide flurochloridone) (SEQ ID NO: 6) are co-transformed to Nannochloropsis oculata CS-179 and Isochrysis sp. CS-177 using the transformation methods described above.

### Example 2

### Generation of Synechococcus PCC7002 Expressing the BFP-Azurite Gene Under the Control of the Cyanobacterial rbcLS Promoter

The BFP-azurite sequence (Mena et al., 2006) is artificially synthesized to enhance stability using the published sequence (SEQ ID NO: 1), but with modifications according to the preferred codon usage of Synechococcus PCC7002 (SEQ ID NO: 7) and with the addition of BamHI restriction sites at both ends. The gene is cloned into pGEM-T vector (Promega, Madison, USA) and then transferred into the BamHI site of pCB4 plasmid (Deng and Coleman, 1999) downstream to the Synechococcus PCC 7002 rbcLS promoter (SEQ ID NO:8) and upstream to rbcLS terminator.

Likewise, similar constructs, made based on codon usage of other cyanobacterial species are generated and transformed into these species.

### Example 3

### Generation of Eukaryotic Algae Cells Expressing DsRed

The DsRed gene is artificially synthesized using the published sequence (accession number BAE53441; SEQ ID NO: 9) with modifications according to the codon usage of the green algae C. reinhardtii (SEQ ID NO: 10) and with the addition of BstBI and BamHI restriction sites at its ends. The gene is cloned into pGEM-T vector (Promega, Madison, USA) and then ligated into the BstBI/BamHI restriction sites of pSI103 (Sizova et al., 2001) replacing the aphVIII selectable marker gene, generating the plasmid pSI-DsRed. In this plasmid the DsRed gene is under the control of the Hsp70A/RbcS2 promoter (SEQ ID NO:11) and 3′ RbcS2 terminator. The gene product fluoresces green light to red wavelengths.

The pSI-DsRed plasmid is co-transformed with pSI103 containing the paromomycin resistance gene to C. reinhardtii CW15 (CC-400) and with pSI-PDS plasmid containing the pds gene (conferring resistance to the phytoene desaturase-inhibiting herbicide flurochloridone) to marine algae using the transformation methods described above.

### Example 4

### Generation of Synechococcus PCC7002 Expressing the DsRed Gene Under the Control of the Cyanobacterial rbcLS Promoter

The DsRed gene is artificially synthesized using the published sequence (accession number BAE53441; SEQ ID NO:9) with modifications according to the codon usage of Synechococcus PCC7002 (SEQ ID NO: 12) and with the addition of BamHI restriction sites at both ends. The gene is cloned into pGEM-T vector (Promega, Madison, USA) and then transferred into the BamHI site of pCB4 plasmid (Deng and Coleman, 1999) downstream to the Synechococcus PCC 7002 rbcLS promoter (SEQ ID NO:8) and upstream to rbcLS terminator.

Likewise, similar constructs, based on codon usage of other cyanobacterial species are generated and transformed into these species.

### Example 5

### Screening for Algal/Cyanobacterial Transformants

BFP-azurite transformants are grown on fresh agar plates for 7 days at 25° C. Colonies are transferred at equal concentrations to 200 μl culture media (as described in the “culture conditions” section) in 96-well micro-well plates, and cultured under the conditions described in the “culture conditions” section, until they reach a substantial cell concentration (˜10⁶ BFP fluorescence is excited at ˜380 nm and monitored at the emission of 450 nm. DsRed and other fluorescent proteins are monitored according to their specific excitation and emission spectra.

Cells from cultures producing the highest fluorescent signal are collected and cultured as single cell colonies under 380 nm near-UV light (duration and intensity are set at LD99% of wild type cells). Surviving cells are then transferred for future culturing and further examination.

### Example 6

### Screening for Transformants Expressing High Level of BFP, DsRed or other Fluorescent Proteins Using Western Immunoblotting

Proteins from transformed algae and cyanobacteria cells with detectable levels of blue or other fluorescence are isolated from algae/cyanobacteria cells utilizing a buffer containing 750 mM Tris pH 8.0, 15% sucrose (wt/vol), 100 μM β-mercaptoethanol and 1 mM phenylmethylsulfonylfluoride (PMSF). Samples are then centrifuged for 20 min at 13,000×g at 4° C., with the resulting supernatant used for western immunoblotting. Western immunoblotting is carried out as described by Cohen et al. (1998) using an anti-RCFP polyclonal Pan antibody primary antibody (Clontech, Palo Alto, Calif., USA) and an alkaline phosphatase-labeled goat anti-rabbit secondary antibody (Sigma). This polyclonal antibody recognizes the GFP-like family of proteins.

### Example7

### Enhanced Photosynthetic Activity

Experimental Design

One of the major goals in the field of production of photosynthetically generated materials (such as oils, proteins, pigments and pharmaceuticals and other co-products) is to utilize the whole spectrum of light reaching the photosynthetic cell, thus increasing photosynthetic efficiency and decreasing heating. In order to demonstrate that cells expressing synthetic genes encoding fluorescent proteins are more efficient using whole light spectra (PAR and near-UV, or full sunlight) reaching the culture, we compare photosynthetic efficiency of transformed algae or cyanobacteria expressing the BFP-azurite and/or any other single or multiple fluorescent proteins set to their respective wild type cultures.

To assess the contribution of fluorescent proteins to cell photosynthetic efficiency, cells are illuminated with a narrow band light with a peak at excitation wavelength of the specific fluorescent protein. Photosynthetic activity of the transgenic algae is examined and compared to that of wild type cells. Oxygen evolution in the light and oxygen consumption in the dark is measured using Clark type electrodes (Pasco Scientific, Roseville, Calif., USA).

Algae and cyanobacteria expressing BFP-azurite have increased photosynthetic activity as measured by oxygen evolution. Significant differences between oxygen evolution of algae and cyanobacteria expressing BFP-azurite and that of their respective wild type are observed when cells are illuminated with light at the excitation wavelength of BFP.

### Example 8

### Enhanced Overall Growth Rate

In order to test that cells expressing synthetic genes encoding fluorescent proteins are more efficient at outdoor light conditions namely, ambient sunlight we compare growth rates of cultures expressing the BFP-azurite to that of wild type cells.

Growth rate at ambient conditions is determined by measuring culture density daily for a period of ten days.

Growth rate is measured using:

Direct cell count

Optical density—at relevant wavelength (e.g. 750 nm)

Pigment/chlorophyll concentration.

Algae and cyanobacteria expressing BFP-azurite have increased photosynthetic activity and growth rate when compared to the wild type.

### REFERENCES

Chisti Y (2007) Biodiesel from microalgae. Biotechnol Adv 25: 294-306
Chow K, Tung W (1999) Electrotransformation of Chlorella vulgaris. Plant Cell Reports 18: 778-780
Cohen A, Yohn C B, Bruick R K, Mayfield S P (1998) Translational regulation of chloroplast gene expression in Chlamydomonas reinhardtii. Methods Enzymol 297: 192-208
Davies J P, Yildiz F H, Grossman A R (1994) Mutants of Chlamydomonas with aberrant responses to sulfur deprivation. Plant Cell 6:53-63
Davies J P, Yildiz F H, Grossman A R (1996) Sacl, a putative regulator that is critical for survival of Chlamydomonas reinhardtii during sulfur deprivation. EMBO 15: 2150-2159.
Deng M D, Coleman J R (1999) Ethanol synthesis by genetic engineering in cyanobacteria. Appl Environ Microbiol 65: 523-528
Gilmore A M, Larkum A W, Salih A, Itoh S, Shibata Y, Bena C, Yamasaki H, Papina M, Van Woesik R (2003) Simultaneous time resolution of the emission spectra of fluorescent proteins and zooxanthellar chlorophyll in reef-building corals. Photochem Photobiol 77: 515-523
Grzebyk, D., O. Schofield, P., Falkowski, and J. Bernhard (2003) The Mesozoic radiation of eukaryotic algae: the portable plastid hypothesis. J. Phycol. 39:259-267)
Guillard, R. R. and Ryther, J. H. (1962). Studies on marine planktonic diatoms. I. Cyclotella nana Hustedt and Detonula confervacaea (Cleve) Gran. Canadian Journal of Microbiology 8: 229-239
Heim R, Prasher D C, Tsien R Y (1994) Wavelength mutations and posttranslational autoxidation of green fluorescent protein. Proc Natl Acad Sci USA 91: 12501-12504
Kindle K L (1990) High-frequency nuclear transformation of Chlamydomonas reinhardtii. Proc Natl Acad Sci USA 87: 1228-1232
Mena M, Treynor T, Mayo S, Daugherty P (2006) Blue fluorescent proteins with enhanced brightness and photostability from a structurally targeted library. Nature Biotechnology 24: 1569-1571
Prokop A, Quinn M F, Fekri M, Murad M, Ahmed S A (1984) Spectral shifting by dyes to enhance algae growth. Biotechnol Bioeng 26: 1313-1322
Salih A, Larkum A, Cox G, Kuhl M, Hoegh-Guldberg O (2000) Fluorescent pigments in corals are photoprotective. Nature 408: 850-853
Sinha R P, Hader D-P (2007) UV-protectants in cyanobacteria. Plant Science 174: 278-289
Sizova I, Fuhrmann M, Hegemann P (2001) A Streptomyces rimosus aphVIII gene coding for a new type phosphotransferase provides stable antibiotic resistance to Chlamydomonas reinhardtii. Gene 277: 221-229
Wyman M, Gregory R P F, and Carr N G (1985) Novel role for phycoerythrin in a marine cyanobacterium, Synechococcus strain DC2. Science 230, 818-820.

What is claimed is:

1. A method to improve photosynthetic efficiency of algal or cyanobacterial cells, said method comprising the step of:
transforming the algal or cyanobacterial cells with a polynucleotide sequence encoding a fluorescent protein capable of absorbing UV- and near-UV or other wavelengths of light poorly used by photosynthetic pigments, and
said polynucleotide sequence being operably linked to a constitutive promoter sequence, whereby the fluorescent protein absorbs UV- and near-UV wavelengths or other wavelengths of light poorly used by photosynthetic pigments and emits wavelengths that are used by photosynthetic pigments.

2. The method of claim 1, wherein the algal cells are selected from the group consisting of Chlamydomonas reinhardtii, Pavlova lutheri, Isochrysis sp. CS-177, Nannochloropsis oculata CS-179, Nannochloropsis like CS-246, Nannochloropsis salina CS-190, Tetraselmis suecica, Tetraselmis chuii and Nannochloris sp.

3. The method of claim 1, wherein the fluorescent protein is BFP-azurite or DsRed protein.

4. The method of claim 3, wherein the cells are Chlamydomonas reinhardtii cells and the BFp-azurite is encoded by SEQ ID NO:2 or SEQ ID NO:3.

5. The method of claim 4, wherein the cells are Chlamydomonas reinhardtii cells and the DsRed protein is encoded by SEQ ID NO:10.

6. The method of claim 4, wherein the sequence is under Hsp70A/RbcS2-promoter.

7. The method of claim 1, wherein the cyanobacterial cells are selected from the group consisting of Synechococcus PCC7002, Synechococcus WH-7803, and Thermosynechococcus elongatus BP-1.

8. The method of claim 7, wherein the fluorescent protein is BFP-azurite or DsRed protein.

9. The method of claim 8, wherein the cells are Synechococcus PC7002 cells and the BFP-azurite protein is encoded by SEQ ID NO:7.

10. The method of claim 8, wherein the cell is Synechococcus C7002, and the DsRed protein is encoded by SEQ ID NO:12.

11. The method of claim 9, wherein the sequence is under rbcLS promoter.

12. The method of claim 1, wherein the algal or cyanobacterial cells are transformed with more than one polynucleotide sequence encoding a fluorescent protein.

13. The method of claim 12, wherein two polynucleotide sequences are transformed in tandem.

14. The method of claim 13, wherein the polynucleotide sequences encode BFP-azurite and DsRed proteins.

15. A transgenic algal or cyanobacterial cell expressing at least one fluorescent protein, wherein at least one fluorescent protein absorbs UV- and near-UV wavelengths or other wavelengths of light poorly used by photosynthetic pigments, and emits wavelengths that are used by photosynthetic pigments.

16. The transgenic algal or cyanobacterial cell of claim 15, wherein the fluorescent proteins are selected from the group consisting of BFP-azurite and DsRed proteins.

17. The transgenic algal cell of claim 16, wherein the cell is selected from the group consisting of Chlamydomonas reinhardtii, Pavlova lutheri, Isochrysis sp. CS-177, Nannochloropsis oculata CS-179, Nannochloropsis like CS-246, Nannochloropsis salina CS-190, Tetraselmis suecica, Tetraselmis chuii and Nannochloris sp.

18. The transgenic algal cell of claim 17, wherein the cell is Chlamydomonas reinhardtii cell and the BFP-azurite protein is encoded by SEQ ID NO: 2 or SEQ ID NO: 3 and the DsRed protein is encoded by SEQ ID NO:10.

19. The transgenic cyanobacterial cell of claim 16, wherein the cell is selected from the group consisting of Synechococcus PCC7002, Synechococcus WH-7803, and Thermosynechococcus elongatus BP-1.

20. The transgenic cyanobacterial cell of claim 19, wherein the cell is Synechococcus PCC7002 cell and the BFP-azurite protein is encoded by SEQ ID NO:7 and the DsRed protein is encoded by SEQ ID NO: 12.

21. The transgenic algal or cyanobacterial cell of claim 15, wherein the cell additionally is transformed with a gene of interest.

22. The transgenic algal or cyanobacterial cell of claim 21, wherein the gene of interest encodes for herbicide resistance, improved oil content or pharmaceutical compounds.

23. The transgenic algal or cyanobacterial cell of claim 22, wherein the gene of interest encodes for resistance to flurochloridone or other phytoene desaturase inhibitors.


SEQUENCE LISTING (12 sequences)

SEQ ID NO: 1 - protein, 238 aa, artificial
  chemically synthesized
MSKGEELFTGVVPILVELDGDVNGHKFSVSGEGEGDATYGKLTLKFICTTGKLPVPWPTLVTTLSHGVQCFSRYPDHMKQHDFFKSAMPEGYVQERTIFFKDDGNYKTRAEVKFEGDTLVNRIELKGIDFKEDGNILGHKLEYNFNSHNIYIMADKQKNGIKVNFKIRHNIEDGSVQLADHYQQNTPIGDGPVLLPDNHYLSTQSALSKDPNEKRDHMVLLEFRTAAGITHGMDELYK

SEQ ID NO: 2 - DNA, 717 bp, artificial
  Chemically synthesized
atgtccaaaggtgaagaactcttcaccggtgtggttccgattctggtcgaactggacggagatgttaatggacataagtttagtgttagcggagaaggagagggtgacgctacctacggcaagctcaccctcaagttcatctgtactaccggcaaattgcctgtcccctggcccacgctcgtgaccaccctttcccacggtgtgcaatgcttttcgcgctatcccgatcacatgaagcagcacgatttctttaagtccgccatgcccgagggatacgtccaagaacgaaccatcttcttcaaggatgatggtaactacaaaacgcgtgccgaagtgaagtttgagggagacacattggtcaaccgcattgagcttaagggtatcgatttcaaggaggacggcaacatcttgggccataaacttgaatacaactttaactctcataacatctacattatggcggacaaacagaagaatggcattaaggtcaacttcaagattcgtcataacattgaggacggatcggtccagttggccgaccactatcagcagaacactccgatcggcgacggacctgtcttgcttccagataatcactacctctcgacacaaagcgccctcagtaaggacccgaatgaaaagcgcgaccacatggttttgttggaatttcgcactgccgctggtattacgcacggcatggatgaactgtacaagtaa

SEQ ID NO: 3 - DNA, 717 bp, artificial
  chemically synthesized
atgagcaagggcgaggagctgttcaccggcgtggtgcccatcctggtggagctggacggcgacgtgaacggccacaagttcagcgtgtccggcgagggcgagggcgacgccacctacggcaagctgaccctgaagttcatctgcaccaccggcaagctgcccgtgccctggcccaccctggtgaccaccctgagccacggcgtgcagtgcttcagccgctaccccgaccacatgaagcagcacgacttcttcaagagcgccatgcccgagggctacgtgcaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggccacaagctggagtacaacttcaacagccacaacatctacatcatggcggacaagcagaagaacggcatcaaggtgaacttcaagatccgccacaacatcgaggacggcagcgtgcagctggcggaccactaccagcagaacacccccatcggcgacggcccggtgctgctgcccgacaaccactacctgagcacgcagagcgccctgagcaaggaccccaacgagaagcgcgaccacatggtgctgctggagttccgcaccgccgccggcatcacccacggcatggacgagctgtacaagtaa

SEQ ID NO: 4 - DNA, 20 bp, artificial
  chemically synthesized
ctggacggagatgttaatgg

SEQ ID NO: 5 - DNA, 20 bp, artificial
  chemically synthesized
tcggagtgttctgctgatag

SEQ ID NO: 6 - DNA, 1743 bp, artificial
  chemically synthesized
atgaccgtggcccgcagcgtggtggccgtgaacctgagcggcagcctgcagaaccgctaccccgccagcagcagcgtgtcctgcttcctgggcaaggagtaccgctgcaactctatgctgggcttctgcggcagcggcaagctggccttcggcgccaacgccccctactccaagatcgccgccaccaagcccaagccgaagctgcgccccctgaaggtgaactgcatggacttcccgcgcccggacatcgacaacaccgccaacttcctggaggccgccgccctgtcctccagcttccgcaacagcgcccgccccagcaagcccctgcaggtggtgatcgctggcgctggcctggcgggcctgagcaccgccaagtacctggccgacgccggccacatccccatcctgctggaggcccgcgacgtgctgggcggcaaggtggccgcctggaaggacgacgacggcgactggtacgagaccggcctgcacatcttcttcggcgcctaccccaacgtgcagaacctgttcggcgagctgggcatcaacgaccgcctgcagtggaaggagcacagcatgatcttcgccatgcccaacaagcccggcgagttcagccgcttcgacttccccgaggtgctgcccgcccccctgaacggcatctgggccatcctgaagaacaacgagatgctgacctggcccgagaaggtgcagttcgccatcggcctgctgcccgccatgatcggcggccagccctacgtggaggcccaggacggcctgaccgtgcaggagtggatgcgcaagcagggcgtgcccgaccgcgtgaacgacgaggtgttcatcgccatgagcaaggccctgaacttcatcaaccccgacgagctgtccatgcagtgcatcctgatcgccctgaaccacttcctgcaggagaagcacggcagcaagatggccttcctggacggcaacccccccgagcgcctgtgcaagccgatcgccgaccacatcgagagcctgggcggccaggtgatcctgaacagccgcatccagaagatcgagctgaacgccgacaagagcgtgaagcacttcgtgctgaccaacggcaacatcatcaccggcgacgcctacgtgttcgccacccccgtggacatcctgaagctgctgctgcccgaggactggaaggagatcagctacttcaagaagctggacaagctggtcggcgtgcccgtgatcaacgtgcacatctggttcgaccgcaagctgaagaacacctacgaccacctgctgttcagccgcagccccctgctgtccgtgtacgccgacatgtccgtgacctgcaaggagtactacaaccccaaccagagcatgctggagctggtgttcgcccccgccgagaagtggatcagctgctccgacagcgagatcatcaacgccaccatgcaggagctggccaagctgttccccgacgagatcagcgccgaccagagcaaggccaagatcctgaagtaccacgtggtcaagaccccccgcagcgtgtacaagaccgtgcccgactgcgagccctgccgccccctgcagcgcagccccatcgagggcttctacctggccggcgactacaccaagcagaagtacctggcgagcatggagggcgccgtgctgtccggcaagctgtgcgcccaggccatcgtgcaggactgctccctgctggccagccgcgtgcagaagtccccccagaccctgaccatcgcctaa

SEQ ID NO: 7 - DNA, 732 bp, artificial
  chemically synthesized
atgtctaaaggcgaagaactctttaccggcgtggtgcccattctcgtggaactcgatggcgatgtgaatggccacaaattttctgtgtctggcgaaggcgaaggcgatgccacctatggcaaactcaccctcaaatttatttgtaccaccggcaaactccccgtgccctggcccaccctcgtgaccaccctctctcacggcgtgcaatgtttttctcgctatcccgatcacatgaaacaacacgatttttttaaatctgccatgcccgaaggctatgtgcaagaacgcaccattttttttaaagatgatggcaattataaaacccgcgccgaagtgaaatttgaaggcgataccctcgtgaatcgcattgaactcaaaggcattgattttaaagaagatggcaatattctcggccacaaactcgaatataattttaattctcacaatatttatattatggccgataaacaaaaaaatggcattaaagtgaattttaaaattcgccacaatattgaagatggctctgtgcaactcgccgatcactatcaacaaaatacccccattggcgatggccccgtgctcctccccgataatcactatctctctacccaatctgccctctctaaagatcccaatgaaaaacgcgatcacatggtgctcctcgaatttcgcaccgccgccggcattacccacggcatggatgaactctataaacaccaccaccaccaccac

SEQ ID NO: 8 - DNA, 304 bp, Synecochoccus
  promoter (1)..(304): Synecochoccus PCC7002 rbcLS promoter
gaattcgagcgggattttatggcttttttaggtatttttgtaagggtaaaataggcccatcaaacagcattagaaatgctaatcagcccaaaaaacaaaagcaatctttttttgttgctaaaagataaaaataagtcgaggctgtggtaacatatcccacagattaaagaaagtcataagacttgaatcttcagaattttaaaaagcagttttgccaacgtaagatttttgaagttttcgaccaacaataccgttactggtatttgtctgttaaagataagcatttttgctggaggaaaacccc

SEQ ID NO: 9 - protein, 238 aa, Discosoma sp
  PEPTIDE (1)..(238): DsRed protein
MKLASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFLRSRAPPPPPLT

SEQ ID NO: 10 - DNA, 714 bp, artificial
  chemically synthesized
atgaagctggccagcagcgagaacgtgatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgagcccccagttccagtacggcagcaaggtgtacgtgaagcaccccgccgacatccccgactacaagaagctgagcttccccgagggcttcaagtgggagcgcgtgatgaacttcgaggacggcggcgtggccaccgtgacccaggacagcagcctgcaggacggctgcttcatctacaaggtgaagttcatcggcgtgaacttccccagcgacggccccgtgatgcagaagaagaccatgggctgggaggccagcaccgagcgcctgtacccccgcgacggcgtgctgaagggcgagacccacaaggccctgaagctgaaggacggcggccactacctggtggagttcaagagcatctacatggccaagaagcccgtgcagctgcccggctactactacgtggacgccaagctggacatcaccagccacaacgaggactacaccatcgtggagcagtacgagcgcaccgagggccgccaccacctgttcctgcgcagccgcgcccccccccccccccccctgacc

SEQ ID NO: 11 - DNA, 702 bp, Chlamydomonas reinhardtii
  promoter (1)..(702): Hsp70A/RbcS2 promoter
gacggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccactccgccctccccggtgctgaagaatttcgaa

SEQ ID NO: 12 - DNA, 714 bp, artificial
  chemically synthesized
atgaaactcgccagtagtgaaaatgtgattaccgaatttatgcgctttaaagtgcgcatggaaggtaccgtgaatggtcatgaatttgaaattgaaggtgaaggtgaaggtcgcccctatgaaggtcataataccgtgaaactcaaagtgaccaaaggtggtcccctcccctttgcctgggatattctcagtccccaatttcaatatggtagtaaagtgtatgtgaaacatcccgccgatattcccgattataaaaaactcagttttcccgaaggttttaaatgggaacgcgtgatgaattttgaagatggtggtgtggccaccgtgacccaagatagtagtctccaagatggttgttttatttataaagtgaaatttattggtgtgaattttcccagtgatggtcccgtgatgcaaaaaaaaaccatgggttgggaagccagtaccgaacgcctctatccccgcgatggtgtgctcaaaggtgaaacccataaagccctcaaactcaaagatggtggtcattatctcgtggaatttaaaagtatttatatggccaaaaaacccgtgcaactccccggttattattatgtggatgccaaactcgatattaccagtcataatgaagattataccattgtggaacaatatgaacgcaccgaaggtcgccatcatctctttctccgcagtcgcgcccccccccccccccccctcacc